(19) United States
(12) Patent Application Publication
(10) Pub. No.: US 2011/0054398 A1
(43) Pub. Date: Mar. 3, 2011

(54) Medical syringe with retractable needle

(76) Inventors: Ilija Djordjevic, East Granby, CT (US); Rahul K. Kanwar, West Hartford, CT (US)

(21) Appl. No.: 12/583,882

(22) Filed: Aug. 27, 2009

(51) Int. Cl. A61M 5/50

(52) U.S. Cl. 604/110

(57) ABSTRACT

A piston in a syringe barrel has a cavity that opens toward the distal end of the barrel. A needle assembly has a hub, a hollow needle fixed in the hub, and a spring seated between the distal end of the barrel and the hub. A membrane is between the hub and the opening of the piston. A locking member is lodged between the barrel and the needle assembly, holding the needle assembly within the barrel to compress the spring. The piston is displaceable through functional positions in which the piston (a) forces serum out through the needle, (b) urges the membrane against the hub, and (c) dislodges the locking member thereby releasing the needle assembly such that the spring displaces the hub and at least a portion of the membrane into the cavity and withdraws the needle tip into the distal end of the barrel.

### BACKGROUND

The present disclosure relates to medical syringes and more particularly to a safety syringe with retractable needle.

According to present statistics, a high number of accidental injuries of medical personnel are caused by the sharp exposed tip of a syringe needle, after injection of the patient. Such needle may be highly contaminated and result in sickness or disease to the medial personnel beyond the physical discomfort of the needle penetration.

Syringes with retracting needles which allow for safe and easy disposal of the syringe while preventing reuse, are known. One example is described in U.S. Pat. No. 6,010,486 “Retracting Needle Syringe”. According to that device, a spring-preloaded needle carrier is triggered to retract into the piston at the end of the piston stroke. The device described in U.S. Pat. No. 6,010,486 is, however, somewhat complicated and difficult to manufacture and assemble. Accordingly, there is a need for a lower cost alternative to that design.

### SUMMARY

The present in invention provides numerous advantages relative to known syringes having retractable needles. The syringe can be manufactured with a reduced part count and simplified functionality of the components because of combined locking and sealing functions. The parts are of a design that can be easily mass produced and automatically assembled. Only minimal changes would be required to conventional tooling for the manufacturer of syringes. Moreover, no special user training is required.

In general, the present disclosure is directed to a retracting needle syringe of the type comprising a syringe barrel having a longitudinal axis and proximal and distal ends; a piston coaxially disposed in the barrel, having proximal and distal ends and a cavity with an opening toward the distal end. A needle assembly is situated within the distal end the barrel, having a hub, a hollow needle fixed in the hub with a tip extending forward of the barrel, and a spring seated between the distal end of the barrel and the hub, whereby a serum volume is defined between the hub and the distal end of the piston. A membrane is interposed between the hub and the opening at the distal end of the piston. A locking member is lodged between the barrel and the needle assembly, thereby holding the needle assembly within the distal end of the barrel to compress the spring while the needle projects from the barrel. With serum in the serum volume and the piston in a proximally retracted position, the piston is manually displaceable toward the needle assembly through functional positions in which the piston (a) forces the serum out of the serum volume through the needle, (b) urges the membrane against the hub, and (c) dislodges the locking member thereby releasing the needle assembly such that the spring displaces the hub and at least a portion of the membrane into the cavity and withdraws the needle tip into the distal end of the barrel.

In one embodiment, the hub has a piercing surface facing the opening at the distal end of the piston, and the membrane covers the opening. Preferably, the functional positions (a), (b), and (c) are sequential, such that between functional positions (b) and (c) of the piston, the piercing surface severs the membrane. Initially, the locking member is in a deformed state bearing on the periphery at the proximal end of the hub. A pusher on the piston is axially aligned with the locking member and displaceable along the periphery of the hub. This displacement drives the piercing surface through the membrane and thereafter dislodges the locking member thereby releasing the needle carrier into the cavity.

In another embodiment, the locking member is in a deformed state at the distal end of the barrel, bearing on the periphery of the distal end of the hub. The pusher is axially aligned with the hub, whereby displacement of the pusher surface to the actuation position urges the hub distally, such that the hub dislodges the locking member.

### BRIEF DESCRIPTION OF THE DRAWING

FIG. 1 is a longitudinal section view of a syringe according to an aspect of the present invention, as taken out of a sterile storage pouch;

FIG. 2 is an enlarged view of the distal region of the syringe of FIG. 1;

FIG. 3 is a schematic representation of the position of an alternative piston within the barrel, ready for filling with serum after the protective cover has been removed from the needle;

FIG. 4 shows the position of the piston within the barrel, as the forward chamber is filled with serum prior to injection;

FIG. 5 is an enlarged view of the distal region of the barrel showing the moment just before the continued displacement of the piston following completion of the injection, releases a locking member on the needle carrier;

FIG. 6 shows the displacement of the needle carrier into the central cavity of the piston by a compression spring seated between the nose of the barrel and the needle carrier, after release of the locking member shown in FIG. 5;

FIG. 7 shows a first alternative embodiment of the needle carrier having a cup-shaped face with sharp rim for penetrating a membrane barrier covering the cavity at the distal end of the piston;

FIG. 8 shows a second alternative embodiment of the needle carrier;

FIG. 9 shows the orientation of the retracted needle carrier associated with the embodiment of FIG. 8;

FIG. 10 shows another embodiment, in which the barrier covering the cavity at the distal end of the piston has dedicated weakened areas for penetration by the needle carrier during the retraction mode;

FIG. 11 shows yet another embodiment of the barrier covering the cavity at the distal end of the piston;

FIG. 12 represents a series of steps in the assembly of a syringe according to the embodiment of FIGS. 1-10;

FIG. 13 shows the steps for completing a piston sub-assembly to be inserted into the barrel sub-assembly according to FIG. 12;

FIG. 14 shows the last three steps of the assembly sequence of FIG. 12 in greater detail;

FIG. 15 shows in greater detail, the steps for installing the disk within the barrel;

FIG. 16 is a view similar to FIG. 3, for an embodiment having a pre-filled vial of serum within the syringe;

FIG. 17 is a detail view of the needle assembly associated with FIG. 16, locked in place at the nose of the barrel;

FIG. 18 shows the locking member associated with FIG. 17 in the compressed and uncompressed conditions;

FIG. 19 shows an alternative disk for use in the embodiment of FIGS. 1-15, incorporating an aspect of the embodiment of FIG. 17; and

FIG. 20 shows the distal end of another embodiment of the syringe with an alternative disk having a reduced diameter.

### DETAILED DESCRIPTION

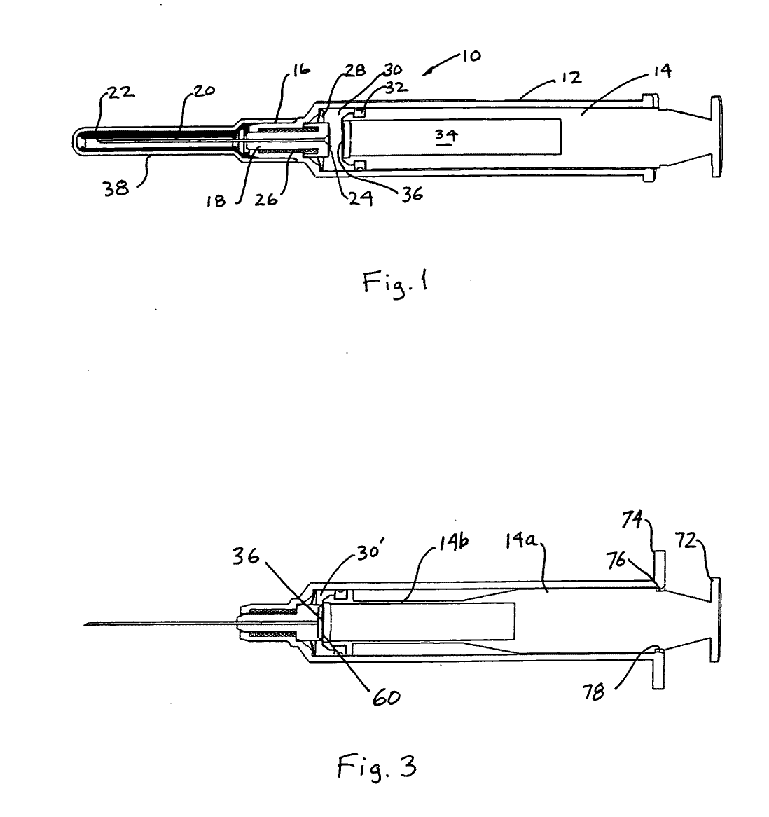

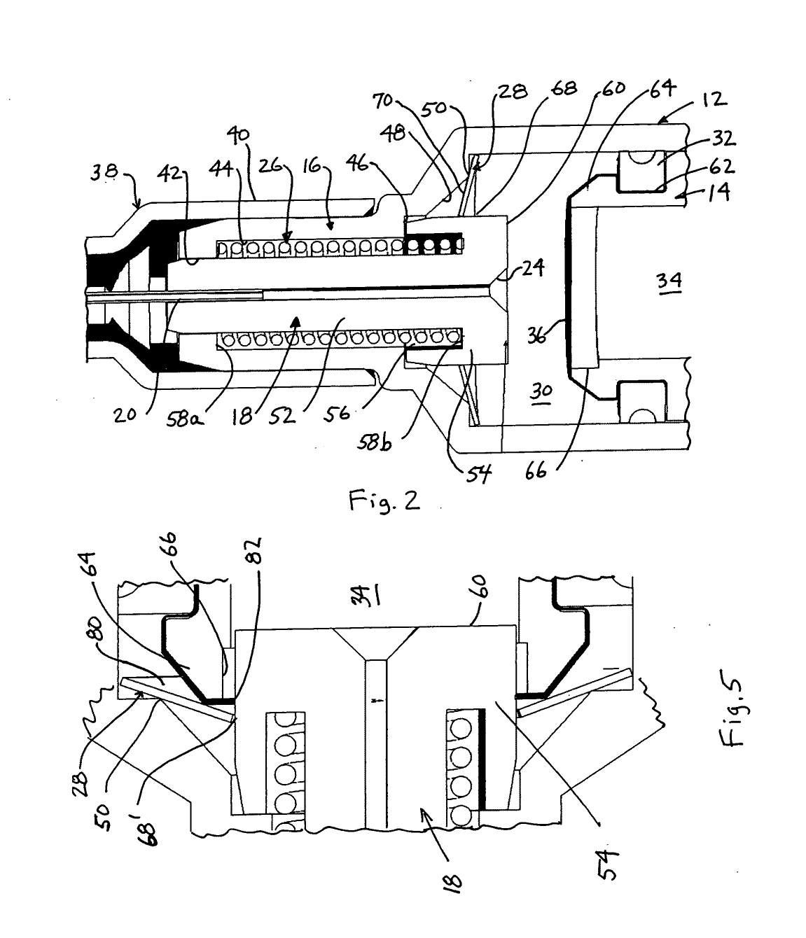

FIGS. 1 and 2 show a syringe 10 having a substantially cylindrically hollow barrel 12 with a piston 14 situated therein that enters the proximal end of the barrel and extends forward to a location spaced from the reduced diameter nose portion 16 at the distal end of the barrel. A needle carrier 18 is situated primarily in the nose 16 of the barrel, and has a hollow needle 20 extending distally to a tip 22. The carrier 18 has a generally funnel-shaped inlet 24 at its proximal end, which may be part of the needle or provide the inlet to a passageway within the carrier 18 in fluid communication with needle 20 affixed within the needle carrier. A helical spring 26 is in compression between the nose 16 and the carrier 18, held in the compressed state by a lock member 28 engaging the carrier 18 and the barrel 12.

In the condition shown in FIGS. 1 and 2, where the syringe has been taken out of the sterile storage pouch, a relatively small chamber 30 is formed between the carrier 18 and spaced apart distal end of piston 14. The piston has an axially extending cylindrical cavity 34 with a distal opening that is sealed by a frangible barrier 36. An annular fluid seal 32 is situated between the piston and the barrel.

The protective cover 38 can be readily removed by pulling it axially, thereby disengaging the frictionally held base 40 of the cover, off the outside of nose 16, thereby exposing the needle 20 just prior to use on the subject.

As is more evident in FIG. 2, the nose 16 of the barrel is also substantially cylindrical, but with a small diameter distal end 42, followed by a larger diameter, longitudinally extending bore 44 which enlarges to form a proximal shoulder 46, and which then tapers outwardly at 48 in the proximal direction, to define an annular support surface 50 facing piston 14.

The needle carrier 18 has an elongated inner hub 52 which at its proximal end is integrated with a larger diameter outer hub 54, thereby defining an annular recess 56 therebetween. The spring 26 is compressed between a distal seat 58A formed by an internal shoulder on the nose 16, and proximal seat 58B at the end wall of recess 56, against the outer hub 54.

The proximal end of the needle carrier 18 defines a piercing surface 60 which, when the needle carrier is released as will be described below, is driven in the proximal direction by the expanding spring 26 to penetrate the frangible barrier 36 such that the needle assembly 18 enters the cavity 34. In the illustrated embodiment, the membrane 36 as installed is a generally cup-shaped piece of rubber or plastic, having a rim portion within an annular notch 62 near the distal end of the piston 14 where it is trapped by an annular seal ring 32, which slidingly bears on the inside diameter of the barrel 12. Preferably, the distal end of the piston is formed as a pusher, and particularly a pushing ring 64, which may have a tapered surface, but in any event is axially aligned with the lock member 28.

The cavity 34 can have a larger diameter opening 66 for facilitating the sliding of the cavity wall over the outside diameter or surface 68 of the outer hub 54, whereby the pusher 64 at the periphery of the barrier 36 can intersect with the locking member 28 as the piston 14 is pushed distally completely through chamber 30.

The lock member 28 is preferably an annular metal or similar washer-like ring or disk with an inner opening having a diameter slightly smaller than the outer diameter 68 of the outer hub 54, compressively deformed at a bend angle oblique to the axis of the syringe as shown at 70. The disk is rigidly supported on its distal side, preferably toward its periphery, at support surface 50, producing a radially inward holding force against the outer hub 54. This press fit at 68 holds the spring 26 in compression as shown in FIG. 2.

FIG. 3 shows the condition of the syringe when ready for filling. This view also shows that the piston 14 may not be of the same diameter over its entire length, but could have a proximal portion 14A that closely conforms to the inner diameter of the barrel and may or may not have wings or other diskrete structure for guiding and thereby facilitating sliding within the barrel, and a proximal portion 14B that is of smaller diameter. The proximal end of the piston 14 has a thumb pad 72, by which the piston 14 can be gently moved from the condition shown in FIGS. 1 and 2 to the condition shown in FIG. 3. The barrier 36 at the distal end of the piston is at or near abutment with the proximal end or piercing face 60 of the needle carrier, thereby establishing a reference volume for the chamber, as indicated at 30′. This position is assured by the engagement of female and male detent members 76, 78 at the proximal end of the barrel, where a flange 74 extends outwardly for convenient engagement by the user's fingers as the thumb pushes on the thumb pad 72.

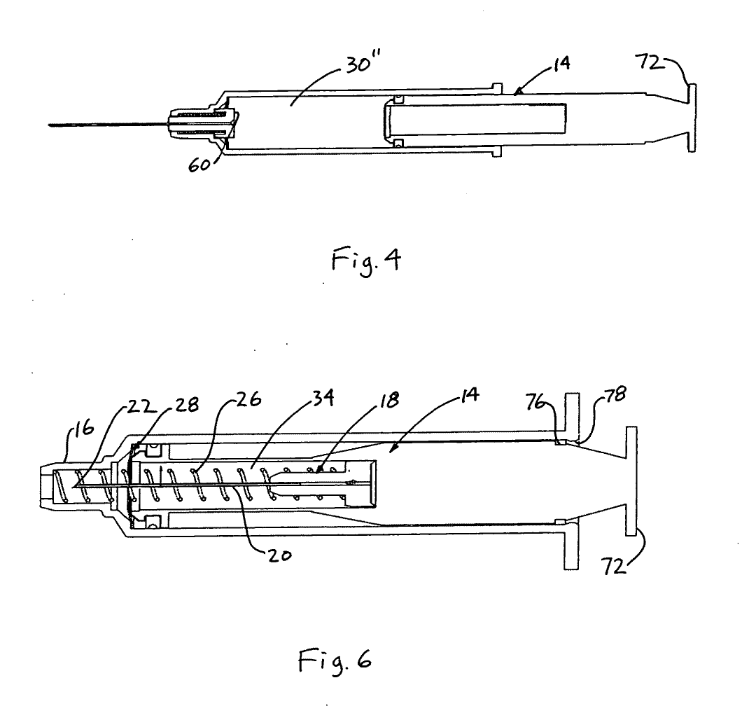

Before injection, the piston 14 is withdrawn by overcoming the detents 76, 78, to a position represented by FIG. 4 thereby filling the chamber 30″ with serum to the extent of the necessary volume as observed by scales (not shown) on the transparent barrel. The needle is then inserted into the patient's flesh and the serum injected by pushing on the thumb tab 72. During such injection, the piston 14 will pass through the position shown in FIG. 2 and reach the position shown in FIG. 5. The push ring 64 travels along the outer diameter of the outer hub 54, with continued displacement resulting in a lever effect by which the contact of the pusher 64 on the proximal surface of the lock member, at a radial position between the interference at 68′ and the support at 50, increases the oblique angle to the extent that the inner end of the lock member loses the radial force against the needle carrier member 18.

Before the pusher ring 64 contacts the lock member 28 the piercing surface 60 on the outer hub has penetrated the barrier 38, such that the proximal end of the carrier 18 enters the cavity 34. The release of the lock member 28 decompresses the spring such that the spring displaces the needle carrier into the cavity and withdraws needle tip 22 into the distal end of the barrel, as shown in FIG. 6.

It is preferable that the axial force of the piston 14 resulting from the user's pressure on the thumb pad break the barrier 36 and produce the initial entry of the needle carrier 18 into the cavity 34. Although the release of the lock member 28 could optionally drive the piercing surface 60 through the barrier and displace the needle carrier into the cavity simultaneously, performing these two functions in sequence minimizes the possibility of the carrier 18 hanging up during the piercing if this piercing function is dependent only on the force of the spring. Tighter tolerance would be needed for maintaining a precise coaxial relationship between the piercing face 60 and the opening of the cavity 34.

It should be appreciated that the locking disk as fabricated is initially completely flat, but because the disk ID is slightly smaller and the OD of the outer hub is slightly larger compared to the respective mating diameters of the needle carrier 18 and the cavity 34, the disk 28 as installed assumes a frusto conical shape. In this position the disk is very stable in supporting both mechanical (skin and tissue penetration by the needle tip) and hydraulic (vacuum during serum filling) coaxial forces. The spring 26 in the fully compressed state provides sufficient force to resist the hydraulic force generated by the relatively low injection pressure. However, when the piston approaches the end position, two events will take place. First, the piercing phase 60 of the needle carrier 18 will penetrate the barrier and open the cavity, and subsequently the pusher 64 will load the disk 28 close to the inner diameter. This will force the disk to pivot around the support 50 and subsequently its inner diameter will expand, thereby releasing the needle carrier 18.

A small clearance at the needle carrier inner hub 52 can be provided which will reverse the above described sequence of releasing and gate opening and at the same time reduce the force required. With the small gap the needle carrier will initially move together with the piston and the only force needed corresponds to the preload of the disk whereas without the gap it must additionally overcome the friction between the needle carrier and disk bore. After the needle carrier is seated the barrier will be penetrated.

It should be further appreciated that during the injection of serum, the inner diameter of disk 28 as press fit on hub 16 at 68 and the circular line contact with the step or shoulder 50 of the barrel form a front seal for chamber 30 whereas the membrane 36 and seal rings such as 32 associated with the piston form a back seal for chamber 30. This assures that all serum in chamber 30 passes through the needle 20. For this reason, the disk 28 is preferably made of a hard rubber or polymeric material such as Polypropylene, Nylon-6 or Acetal.

FIG. 6 shows the piston 14 in its maximum distal insertion within the barrel, and the needle carrier 18 fully within the cavity while the tip of the needle 22 is within the nose 16 of the barrel.

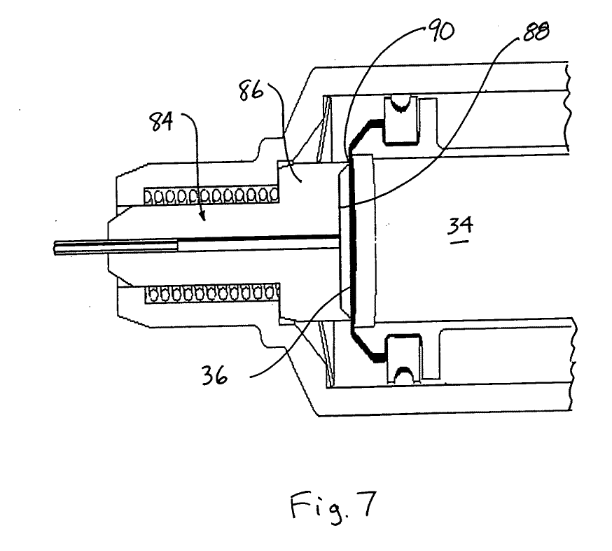

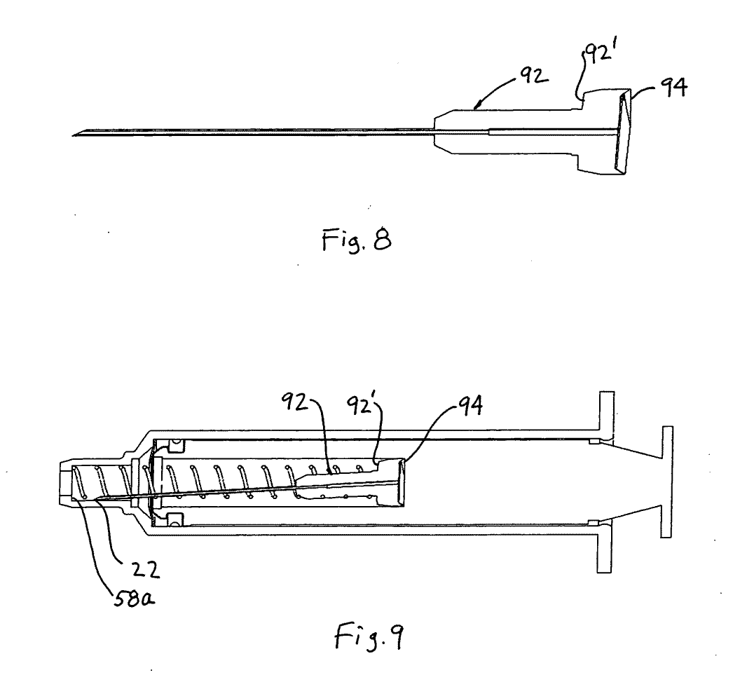

The piercing surface 60 as shown in FIGS. 1 and 2 is simply a flat circle, but as shown in FIG. 7, an alternative carrier 84 has an outer hub 86 with a dish shaped proximal face 88 which includes an annular sharp edge 90 that can more easily pierce the membrane 36.

Whereas in the embodiments shown in FIGS. 6 and 7, the needle carrier 18 and needle 20 remain substantially coaxial with the axis of the syringe after retraction, FIGS. 8 and 9 show another embodiment in which a further safety feature is implemented. In this embodiment, the needle carrier 92 has a dish face 94 at the proximal end, similar to that shown in FIG. 7, except that the dish and cutting edge are angled obliquely relative to the axis. Upon retraction, as shown in FIG. 9, the spring force remains axial and is applied axially against the distal, flat fact of the 92′ of the needle carrier. Because the diameter of the inner hub 92 is smaller than the diameter of the spring, the axial force on face 92′ urges the rim at 94 to square up with the vertical wall of the cavity but due to the angle of the dish 94 the inner hub and its coaxial needle assume an angle relative to the axis. In the event the syringe is accidentally dropped tip down, and the inertial force should overpower the relatively low force of the fully expanded spring, the tip of the needle will hit a solid wall such as seat 58A instead of being re-exposed through the exit bore of the nose.

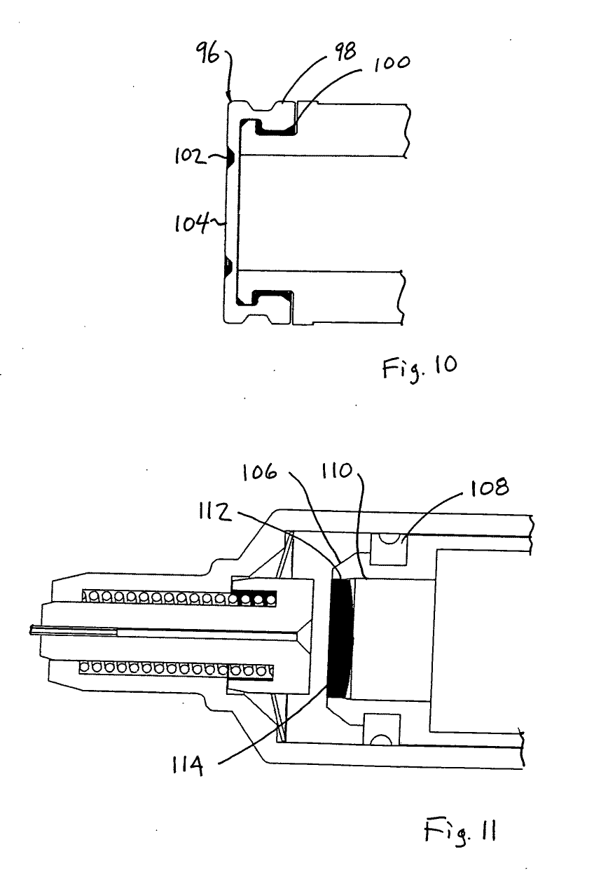

In another embodiment shown in FIG. 10, the membrane 96 serves the dual function of providing the fluid seal 98 between the piston and the barrel, and the barrier 104 at the opening of the cavity. The membrane is substantially cup-shaped, with an enlarged rim portion defining the seal 98 with the radial inner portion mounted in the annular notch 100 immediately behind the push ring portion of the piston. The barrier portion 104 preferably includes weakened circular groove or similar region 102 aligned with the piercing surface of the outer hub 54. It can be appreciated that the membrane has an enlarged rim portion 98 in the notch 100, base portion 104 overlying the push ring and opening thereby constituting the barrier, and weakened region 102 within the piercing surface of the outer hub.

In yet another embodiment, shown in FIG. 11, the barrier is a distinct plug 114 retained by compressive deformation against a cylindrical wall defining the opening at 112 and displaceable by the needle carrier through the cavity as the piston is displaced into overlapping relation with the outer hub. In this embodiment, the push ring 106 is not covered by a membrane and the fluid seal 108 is distinct from the membrane.

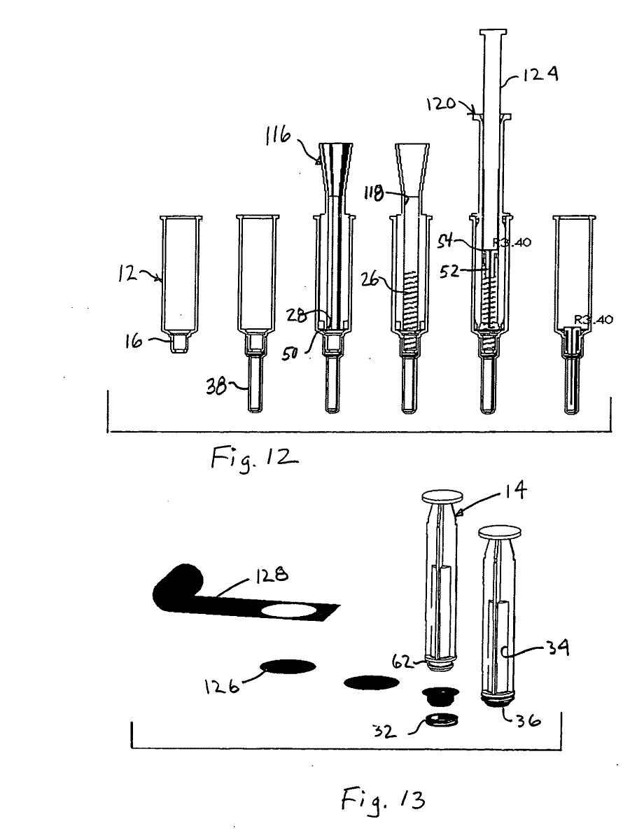

FIGS. 12-14 show the ease with which the syringe such as described previously with respect to FIGS. 1-10, can be readily assembled. With particular reference to FIG. 12, starting from the left, a syringe barrel 12 is selected and a needle protection cover 38 is installed over the nose portion 16 of the barrel. The washer-like locking disk 28 is then placed in the barrel and a special tool 116 is inserted in the barrel to push the locking disk until it reaches the shoulder or step 50 at the bottom of the main portion of the barrel.

As shown in the sequence of FIG. 15, initially, the disk 28 is in the form of a flat washer, at the entrance to the barrel 12, per FIG. 15(a). The disk diameter is preferably slightly larger than the mouth of the barrel especially at the detent 78, but smaller than the main bore of the barrel. In order to move further down into the bore, the disk must deform, per FIGS. 15 (b) and (c). The installation tool 116 contacts the disk at the central hole while the disk is supported closer to the outside diameter. This will cause the disk to bend (basically pivoting around the barrel wall) and once it assumes a conical shape it will continue to slide through the bore per FIG. 15(d) until it reaches the stop or shelf 50 per FIG. 12. Because of the elasticity of the material the central bore will temporarily increase in diameter while the outside diameter will shrink to fit through the narrower bore.

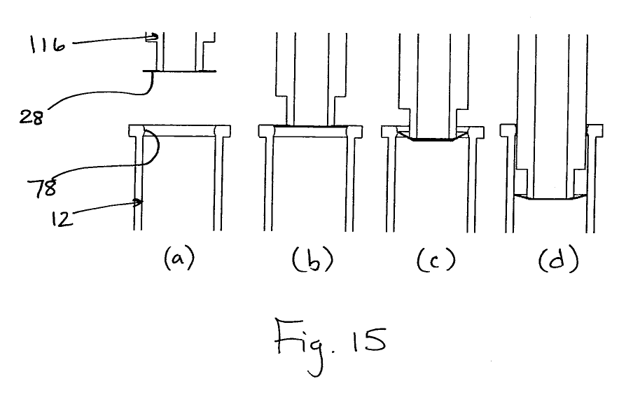

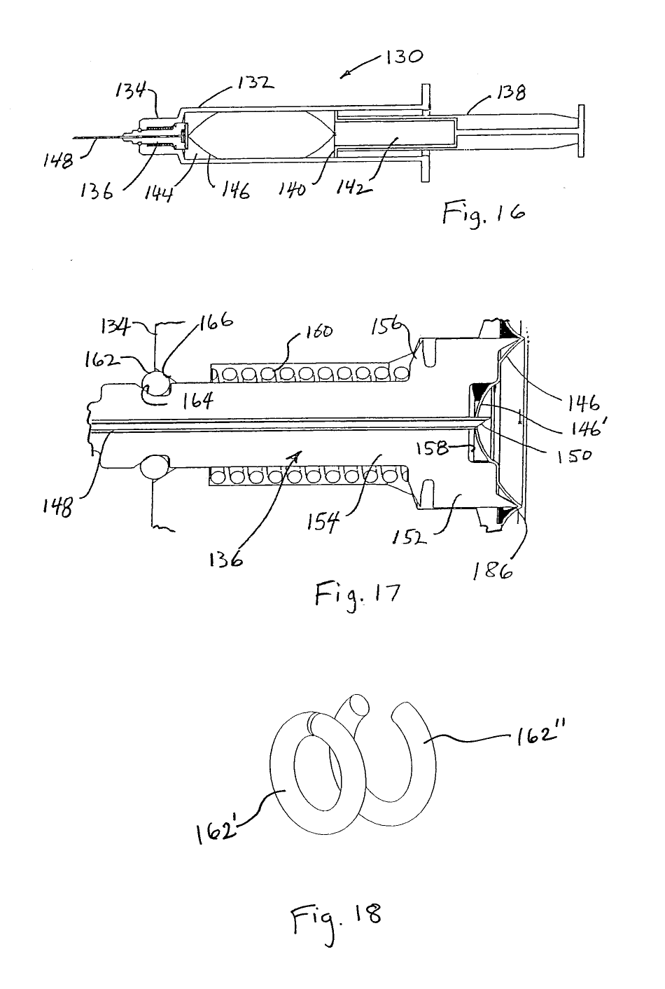

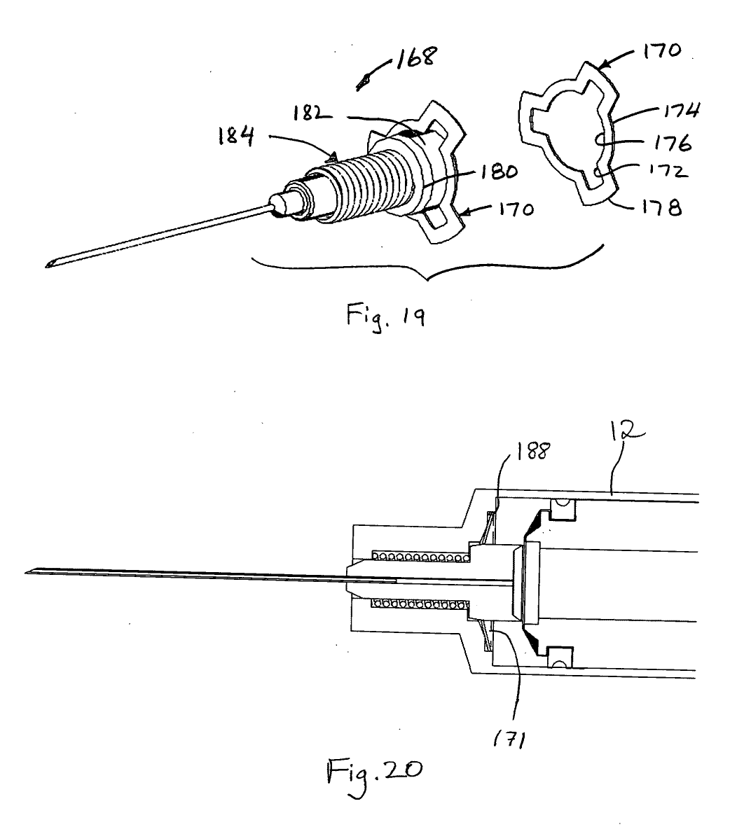

Subsequently, as also shown in FIG. 14(a) the needle carrier return spring 26 is dropped through the central bore 118 of tool 116, whereupon the tool 116 is removed. As also shown in FIG. 14(b) another tool 120 is then inserted into the barrel, while holding the hub 52, 54 of the needle assembly. The hub is held inside the tool by a small protrusion or the like 122 in a position such that the tip 22 of the needle is fully hidden within the main cylindrical bore of the barrel. A push rod 124 is displaced axially through the tool 120, displacing the hub of the needle carrier until it seats on the step or shoulder 46 of the barrel. As previously described, the displacement of the outer hub 54 in the aperture of the locking disk 28 loads the disk and thus captures the needle assembly in the condition shown at (c) of FIG. 14. Once the needle assembly is locked in place, the installation tool 120 and associated push rod 124 can be retracted.

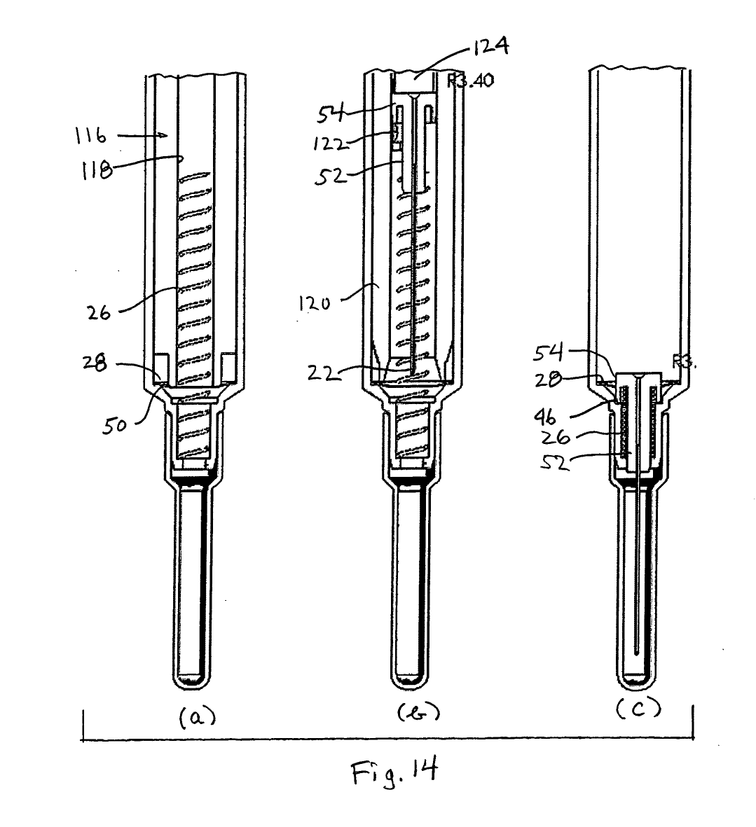

The partially assembled syringe is then ready to receive the piston or plunger, which is preferably assembled as shown in FIG. 13. A round membrane disk 126 is stamped out of a thin plastic band 128. The disk 126 is pulled over the proximal tip of the piston, assuming a dish or cup shape. The rubber seal 32 is then placed over the annular groove 62 thereby providing a membrane closure to the cavity 34 of the piston. The piston with closure is then inserted into the barrel as shown in FIG. 14(c) to arrive at the completed syringe as shown in FIG. 1. Typically, the syringe is placed inside a protective pouch which can be firmly welded and the entire package subject to sterilization.

FIGS. 16-18 show another embodiment of a syringe 130 according to the inventive concept. This embodiment is suitable for use with pre-filled syringes. The syringe has a barrel 132 with nose 134 in which the needle assembly 136 is situated. The piston 138 has a proximal end 140 with axial cavity 142. A serum volume 144 is defined between the piston 140 and the needle assembly 136. In this embodiment, the serum is contained within a closed membrane defining a vial 146 situated in the serum volume 144. Unlike the previously described embodiment, this membrane does not initially cover or seal the cavity 142 relative to the serum volume 144, but as will be described, the membrane defining the vial 146 is frangible, or equivalently deformable, so that all or a portion of the vial enters the cavity 142 along with the needle assembly 136 following injection of the serum to the needle. The preferred configuration of the needle assembly 136 during injection through needle 148 is shown in FIGS. 17 and 18.

As with the previously described embodiments, the piston 138 is manually displaceable toward the needle assembly 136 through several functional positions. First, the piston 138 forces the serum out of the serum volume through needle 148. This is achieved by compressing the vial 146 until the proximal end of the needle 148 penetrates the vial and continued displacement of the piston empties the vial through the needle. During this injection step, the needle assembly 136 remains in a locked position within the barrel nose 134. This is shown in FIG. 17, wherein the portion 146′ of the membrane 146 has been penetrated by the proximal end 150 of needle 148.

The needle assembly 136 has an outer hub 152 and an inner hub 154, as in the previously described embodiment. Preferably, the distal portion 156 of the outer hub 152 defines a seat for spring 160 which is compressed with respect to a distal seat formed in nose 134. The spring 160 as compressed therebetween preloads the needle assembly for displacement toward the piston when the injection has been completed. The proximal end of the outer hub 152 preferably has a central recess 158 from which the proximal end 150 of the needle projects. Upon pressurization of the vial 146, a lobe or the like 146′ conforms to the recess and assures penetration by the needle 150. Preferably, the proximal face of the outer hub 152 defines a piercing surface, preferably with a sharp annular edge such as indicated at 186. This edge does not perforate the membrane 146 during injection.

In the second position the piston urges the membrane 146 against the piercing surface 186 at the conclusion of the injection step. Preferably, the emptied vial is at least partially urged into the piston cavity 142 by the piercing surface 186. In the last step or position, the needle carrier follows at least a portion of the vial into the cavity 142.

During injection and preferably until at least some of the membrane 146 has entered the cavity 142, the needle assembly 136 is held in place by lock member 162 lodged between a tapered or similarly ramped groove 164 near the distal end of the inner hub 154, and a conical or similarly ramped or tapered surface 166 on the distal end of the barrel nose 134. One suitable locking member is shown in FIG. 18. The condition 162′ corresponds to the compressed state as shown in FIG. 17, whereas the condition 162″ is the natural or neutral condition. Thus, when the locking ring is in the condition 162′, it is under radially inward compression, which if released will expand into the condition shown at 162″.

At the same time, or after the injection event, the piston moves the carrier assembly 136 slightly in the distal direction, enabling the compressed locking ring 162 to expand and “jump out” of the groove 164. The spring 160 is now free to act against seat 156, release the needle carrier and displace it into the cavity, thereby withdrawing the needle tip into the distal end of the barrel. The maximum displacement of the piston 138 toward the needle assembly 136 can optionally directly act against the sharp rim 186, thereby cutting a central portion of the membrane out of the vial whereby that central portion of the membrane and the carrier assembly are retracted into the cavity 142.

It should be appreciated that during injection the volume of the vial is reduced corresponding to the reduction in volume of serum contained therein. As a consequence, the membrane begins to deform and eventually collapses. The deformation at the distal end of the vial enables the membrane to enter the recess 158 for penetration by the needle end 150. At the same time, the proximal end of the vial facing the piston also begins to conform to the open end of the cavity 142. The friction between the vial wall and the rand of the piston is sufficient to prevent the vial from completely entering the cavity until the pressure in the vial has dropped to near zero (because of the serum drain). The vial then becomes flaccid and can fold and be pushed by the retracting needle assembly into the piston cavity together with the needle assembly. To facilitate complete entry of the vial into the cavity, a small venting hole can be provided at the proximal end of the piston cavity. Another option is to shape the contact surface between piston and the vial as several concentric rings and by that increase friction to prevent the vial from entering the piston cavity before all or nearly all of the serum has been injected. Another possibility would be to configure and size the cutting edge 186 and the ID of the piston cavity in such a way that when the piston reaches its fully inserted (bottom) position, the vial will be sheared off. With reference to FIG. 20, an additional embodiment exists wherein the metal disk 171 has an outer diameter that is smaller than the inner diameter of the barrel 12 to minimize or eliminate contact between the disk 171 and the barrel surface during installation. Here, the barrel is fit with a recess 188 at the distal end of the chamber 30 for engaging the disk 171.

It should be understood that, as used herein, the “piercing surface” at the proximal end of the needle carrier or hub, performs the broad function of assuring that the membrane passes through (or pierces) the piston cavity opening and thus enters the cavity. In some embodiments, a membrane at the opening is merely cut, whereas in other embodiments all or part of the membrane is pushed into the cavity.

FIG. 19 shows one embodiment 168 of how a metal locking member 170 would preferably be employed in a combination of the concepts discloses in FIGS. 1-15 and 17. With the plastic disk 28 of FIGS. 1-15, the disk is both a locking member and a front seal for the serum volume or chamber 30, to resist the fluid pressure exerted on the serum by the piston during injection. Plastic has the advantage of being somewhat flexible and pliable, facilitating assembly through the barrel and conforming as a seal to the hub 54, but care should be taken to assure that the pressure differentials acting on the disk during drawing of serum and during injection do not excessively deform and thus undermine the operation of the disk. A solid metal disk locking member 170 would provide a stronger locking action to better resist pressure differentials, but due to increased rigidity could be more difficult to install and the force necessary to stretch the inner bore and release the needle carrier 18 could be too high, i.e., outside of the force level applied with normal syringes.

However if the disk 170 is provided with inner and outer cut outs 172, 174, installation would be easier while the stronger locking action would be retained. As used herein, “perforations” encompasses cut outs, holes, etc. The inner diameter edge 176 need not be stretched but merely bent around the shelf 50 in the barrel. In a similar manner the outer diameter edge 178 need not be compressed but only bent about the mating diameter of the installation tool 116 during the assembly. The perforations render the disk pressure insensitive, so it can resist maximum vacuum when serum is drawn and also maximum pressure during injection, without collapsing. Such disk provides excellent locking at reduced installation and actuation forces but such disk does not also provide the fluid seal function at the front of the serum chamber.

As shown in FIG. 19, the front seal 180 in this embodiment can be the same or similar to the front seal 156 in the embodiment of FIG. 17, i.e., a flexible formation carried on the hub 182 of needle assembly 184 and engaging the inside surface of the barrel. This seal 180 can in the alternative be a simple elastomeric insert or “O” ring but, when integrated in the needle carrier as shown, the part count is minimized.

The strongest advantage of this perforated metal disk is that by being absolutely insensitive to internal pressure, it will not release the needle carrier regardless internal pressure, but it will be released by the full distal displacement of the piston.

1. A retracting needle syringe for injecting a serum into flesh, comprising:
a syringe barrel, said barrel having a longitudinal axis and proximal and distal ends;
a piston coaxially disposed in said barrel, having proximal and distal ends and a cavity with an opening toward said distal end;
a needle assembly situated within said distal end of said barrel, said needle assembly having a hub, a hollow needle fixed in the hub with a tip extending forward of the barrel, and a spring seated between the distal end of the barrel and the hub, whereby a serum volume is defined between the hub and the distal end of the piston;
a membrane interposed between the hub and the opening at the distal end of said piston;
a locking member lodged between the barrel and the needle assembly, thereby holding the needle assembly within the distal end of the barrel to compress said spring while the needle projects from the barrel; and
wherein with serum in said serum volume and the piston in a proximally retracted position, the piston is manually displaceable toward the needle assembly through functional positions in which the piston (a) forces the serum out of the serum volume through the needle, (b) urges the membrane against the hub, and (c) dislodges the locking member thereby releasing the needle assembly such that said spring displaces the hub and at least a portion of the membrane into said cavity and withdraws the needle tip into the distal end of the barrel.

2. The syringe of claim 1, wherein the functional positions (a), (b), and (c) are sequential.

3. The syringe of claim 1, wherein the hub has a piercing surface facing the opening at the distal end of the piston, the membrane is interposed between the piercing surface and said opening, and between functional positions (b) and (c) of the piston, the piercing surface severs the membrane.

4. The syringe of claim 1, wherein after the piston dislodges the locking member in functional position (c) the displacement of the needle carrier drives the piercing surface through the membrane.

5. The syringe of claim 1, wherein the locking member is a compressible O ring trapped in a compressed state between the distal end of the barrel and the needle assembly as a result of the spring urging the needle assembly in a proximal direction relative to the barrel

6. The syringe of claim 5, wherein
The hub has a proximal end with a central recess facing the distal end of the piston and the needle has a proximal end projecting into the recess toward the distal end of the piston;
the serum volume contains a vial of serum, said vial defined by said membrane in the form of a closed surface;
during position (a) the piston pushes the vial against the proximal end of the needle whereby the serum is injected through the distal end of the needle and the vial is emptied;
during position (b) the emptied vial is at least partially urged into the piston cavity by the piercing surface; and
during position (c) the needle carrier follows at least a portion of the vial into the cavity.

7. A retracting needle syringe, comprising:
a syringe barrel, said barrel having a longitudinal axis and proximal and distal ends;
a piston coaxially disposed in said barrel, having proximal and distal ends and a cavity with an opening toward said distal end;
a needle assembly situated within said distal end of said barrel, said needle assembly having a hub, a hollow needle fixed within the hub and having a tip extending forward of the barrel, and a spring seated between the distal end of the barrel and the hub, said hub having a piercing surface at a reference axial position spaced from the opening at the distal end of said piston;
a frangible membrane interposed between the piercing surface and the opening at the distal end of said piston;
a locking member lodged between the barrel and the needle carrier, thereby holding the needle assembly within the distal end of the barrel to compress said spring while the needle projects from the barrel; and
a pusher surface at the distal end of the piston, displaceable with the piston through the barrel to an actuation position distally beyond said reference position of the piercing surface, wherein said displacement drives the piercing surface through said membrane and upon reaching the actuation position dislodges the locking member thereby releasing the needle carrier such that said spring displaces the needle carrier into said cavity and withdraws the needle tip into the distal end of the barrel.

8. The retracting needle syringe of claim 7, wherein
the locking member is in a deformed state bearing on the periphery at the proximal end of the hub;
the pusher is axially aligned with the locking member and displaceable along the periphery of the hub;
whereby displacement of the pusher surface to the actuation position along the periphery of the hub dislodges the locking member.

9. The retracting needle syringe of claim 7, wherein
The locking member is in a deformed state at the distal end of the barrel bearing on the periphery of the distal end of the hub;
The pusher is axially aligned with the hub;
whereby displacement of the pusher surface to the actuation position urges the hub distally, such that the hub dislodges the locking member.

10. A retracting needle syringe, comprising:
a syringe barrel for holding liquid, said barrel having a longitudinal axis and proximal and distal ends;
a plunger rod or piston coaxially disposed in said barrel, having proximal and distal ends and a cavity with an opening toward at said distal end, covered by a frangible barrier;
a needle assembly situated within said distal end of said barrel, said needle assembly having a needle carrier including an inner hub, an outer hub, a hollow needle fixed within the inner hub and having a tip extending forward of the barrel, and a spring seated between the distal end of the barrel and the outer hub, said outer hub having a piercing surface facing said barrier,
a locking member press fit between the barrel and the outer hub, thereby holding the needle assembly within the distal end of the barrel to compress said spring while the needle projects from the barrel; and
a pusher at the periphery of the barrier, sized to pass axially around the piercing surface of the outer hub into contact with the locking member as the piston is pushed distally, thereby severing the barrier, releasing the press fit of the locking member, and decompressing the spring such that said spring displaces the needle carrier into said cavity and withdraws the needle tip into the distal end of the barrel.

11. The retracting needle syringe of claim 10, wherein
the barrel has a reduced diameter, hollow nose at the distal end; and
said spring has a distal seat against a shoulder in said nose and a proximal seat in an annular recess between said inner and outer hubs.

12. The retracting needle syringe of claim 10, wherein
the outer hub is cylindrical with a defined outer diameter;
the lock member is an annular disk with an inner opening having a diameter smaller than the outer diameter of the outer hub, a distal side bearing on a support surface in the barrel, and a compressive bend that produces a radially inward holding force against the outer hub.

13. The retracting needle syringe of claim 10, wherein said piercing surface is cup-shaped with a sharp rim.

14. The retracting needle syringe of claim 13, wherein said cavity has a back wall perpendicular to the axis and the piercing member rim is obliquely angled relative to the axis.

15. The retracting needle syringe of claim 10, wherein the distal end of the piston includes a pusher ring in axial alignment with the lock member, an annular notch, and an annular fluid seal seated in the notch and slidingly engaging the barrel.

16. The retracting needle syringe of claim 15, wherein said barrier is a membrane secured in said notch and covering the ring and said opening.

17. The retracting needle syringe of claim 10, wherein
the distal end of the piston includes a pusher ring in axial alignment with the lock member, an annular notch, and a cup shaped cap having an enlarged rim portion in said notch, and base portion overlying said ring and opening and constituting said barrier;
and a weakened region within the piercing surface of the outer hub.

18. The retracting needle syringe of claim 10, wherein said barrier is a plug retained by compressive deformation against a cylindrical wall defining said opening and displaceable through the cavity with the needle carrier.

19. The retracting needle syringe of claim 10, comprising a detent engagement between the proximal end of the syringe barrel and the plunger, which establishes a reference position of the barrier relative to the needle carrier and a reference chamber volume bounded by needle carrier, the lock member, the distal end f the piston, and the barrier.

20. The retracting needle syringe of claim 12, wherein the locking member is a solid disk providing a fluid seal against the hub and against the bore of the barrel.

21. The retracting needle syringe of claim 12, wherein
the locking member is a perforated metal ring having an inner edge bearing on the hub and an outer edge bearing on the bore of the barrel; and
the needle assembly includes a flexible annular formation providing a fluid seal against the bore of the barrel.

22. The retracting needle syringe of claim 12, wherein the perforations on the metal ring are inner and outer cut outs.
